SEQUENCE LISTING (1 sequence)

SEQ ID NO: 1 - DNA, 391 bp, Ganoderma sessile
  source 1..391: mol_type = genomic DNA
cattatcgagttttgactgggttgtagctggcctcccgaggcatgtgcacgccctgctcatccactctacacctgtgcacttactgtgggcttcagacgtcgtgaagcgggctctttacggagcttgtggagcgtgtctgtgcctgcgtttaccacaaactctataaagtattagaatgtgtattgcgatgtaacgcatctatgtacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcatgaaatcttcaacttacaaacctttgcggg

(19) United States
(12) Patent Application Publication
(10) Pub. No.: US 2026/0284099 A1
(43) Pub. Date: Sep. 24, 2026

(54) GANODERMA SESSILE POLYSACCHARIDE MIXTURES, COMPOSITIONS, AND RELATED METHODS

(71) Applicant: FUTURE BIOME CORPORATION, New York, NY (US)

(72) Inventor: Maria Soledad Vela Gurovic, Bahía Blanca, Buenos Aires (AR)

(73) Assignee: FUTURE BIOME CORPORATION, New York, NY (US)

(21) Appl. No.: 19/472,716

(22) PCT Filed: Jun. 27, 2024
(86) PCT No.: PCT/IB2024/056296

§ 371 Date: Oct. 6, 2025

Related U.S. Application Data
(60) Provisional application No. 63/524,483, filed on Jun. 30, 2023.

(51) Int. Cl. A61K 31/715; A61K 9/00; A61P 1/14; C12N 1/14; C12P 19/04; C12R 1/645

(52) U.S. Cl. CPC A61K 31/715; A61K 9/0053; A61P 1/14; C12N 1/14; C12P 19/04; C12R 2001/645

(57) ABSTRACT

The present invention relates to a polysaccharide mixture comprising Ganoderma sessile polysaccharides (GSP), compositions and products that comprise the polysaccharide mixture of the present invention and methods altering the microbiota structure and function such as Short Chain Fatty Acid (SCFA) production by means of the polysaccharide mixture.

This application claims the benefit of priority from the provisional Application No. 63/524,483, filed on Jun. 30, 2023, and the entire contents of that application are incorporated herein by reference.

### FIELD OF THE INVENTION

The present invention relates to fungal polysaccharides mixtures, more particularly to Ganoderma sessile polysaccharides (GSP), a method for obtaining said GSP, a composition comprising said GSP, a method for increasing the Short Chain Fatty Acids (SCFA) production in the gastrointestinal tract, skin, serum, or mucosa of a subject by using said GSP, a method for altering the microbiome structure and/or function present in the gastrointestinal tract, skin, or mucosa, and a method to decrease upper respiratory symptoms of common cold, increase daily activity, decrease stress, improve gastrointestinal quality of life, and balance mood in a subject by using said GSP.

### BACKGROUND

Chronic inflammatory diseases cause more than 50% of all deaths worldwide (Furman et al, 2019). At least one third of the population live with chronic inflammation, symptomatic or not. Systemic chronic inflammation is closely dependent on the gut microbiome and its functionality. Short Chain Fatty Acids (SCFAs) produced by the gut microbiota contribute to reducing inflammation by exerting different mechanisms on immune and epithelial cells of the gut mucosa (Scheithauer et al. 2020).

A better performance of the gut microbiota is also associated to effects on mood, anxiety, stress, and depression. These conditions currently affect 15-20% percent of people (Bear et al. 2020). In a wide extent, these conditions can be modulated by improving dietary habits. This can be achieved, for example, by increasing the intake of prebiotic fiber.

Most of prebiotic fiber is composed by carbohydrates, which can be divided into oligo- and polysaccharides, depending on the number of units of the saccharide chain. Those oligo- and polysaccharides that are not degraded by human digestive processes and are not absorbed by enterocytes are considered dietary fiber.

Homopolysaccharides are composed of a single type of monosaccharide. For example, homoglucan is composed of glucose. β-glucans are homoglucans with glycosidic bonds recalcitrant to human enzymes. β-glucans having different molecular weights and chemical branching degrees are present in cereal, baker's yeast, mushrooms, and seaweed.

Meanwhile, heteropolysaccharides or heteroglycans are composed of more than one type of monosaccharide. These chemical differences confer different health functions such as immunomodulatory, cardiovascular effects and gut health.

It is known in the art that polysaccharides can modify the production of Short Chain Fatty Acids (SCFA) in the microbiota. For example, Inulin is a homopolysaccharide composed of fructose, and it is the first prebiotic to be approved worldwide.

More particularly, it is known that fungi extracts and oligosaccharides can increase the production of SCFA in microbiota, as described in WO 2019/205942 A1, which further describes a method to improve the production of SCFA in gut microbes by the application of a formulation, wherein said formulation comprises oligosaccharides and fungi extracts.

On the other hand, WO 2022/155314 A1 describes the use of a blend of three species of fungi, including Ganoderma lucidum, to produce an increase of butyrate in gut microbes. In the same document, a fungal composition for the modulation of the microbiota comprising β-glucans is disclosed, and it is stated that the composition is used in an effective amount, i.e. capable of producing the desired effects, like reduction, prevention and treatment of diseases.

While the prior art discusses Ganoderma fungi or Ganoderma extracts to modify the production of SCFA in gut microbiota, they do not disclose or teach how to extract or produce polysaccharides from Ganoderma sessile, nor how to produce compositions based on G. sessile polysaccharides (GSP), nor how to produce heteroglycans from either G. sessile or G. lucidum. Further, the prior art does not disclose a proper method to effectively use GSP for the increase of SCFA production in microbiota.

In general, the extraction of polysaccharides from fungi of the Ganoderma genus is known in the art. Zhang et al, 2019, describe a general method to extract polysaccharides from Ganoderma, and Viceconte et al, 2021. describes a method of extraction of polysaccharides specifically from Ganoderma sessile. However, the prior art does not suggest the use of these GSP in a composition, or a method to produce an increase SCFA production in gut, skin, or mucosal microbiota.

There is therefore a need to provide alternative or improved compositions and methods for restoring the balance of microbiota by improving the microbiota structure and or increasing their function including production of Short Chain Fatty Acids (SCFA) in the microbiota, as well as treating or preventing diseases, and improving the quality of life of subjects undergoing subclinical health issues related to dysbiosis and low production of SCFA by the microbiota.

### BRIEF DESCRIPTION OF THE INVENTION

An aspect of the present invention is a polysaccharide mixture, wherein said polysaccharide mixture comprises
at least one polysaccharide extracted from Ganoderma sessile, and
wherein the polysaccharide mixture comprises arabinose, xylose, mannose, galactose, glucose, fucose, and rhamnose as constituting monomers of the polysaccharides comprised therein.

Another aspect of the present invention is a fermentation process for the production of the polysaccharide mixture, wherein said fermentation process takes place in a bioreactor having a volume of 10-10000 L, preferably a volume of 10-1000 L, more preferably a volume of 10-100 L, and said process comprises concentrating the fermentation product by ultrafiltration.

In an embodiment, the fermentation comprises fermenting Ganoderma sessile.

Another aspect of the present invention is a composition, wherein said composition comprises a polysaccharide mixture of the present invention and at least one additional excipient selected from a flavoring agent, an antioxidant, a colorant, a mineral, a vitamin, a pH regulator, a protein, a lipid and carrier, and any combination thereof.

In an embodiment, the composition of the present invention comprises the polysaccharide mixture of the present invention in an amount of at least 0.1% w/w.

Another aspect of the present invention is a health product comprising the polysaccharide mixture of the present invention, and at least one additional excipient suitable for use in a health product.

Another aspect of the present invention is a cosmetic product comprising the polysaccharide mixture of the present invention, and at least one additional cosmetically acceptable excipient.

Another aspect of the present invention is a dietary supplement comprising the polysaccharide mixture of the present invention, and at least one additional excipient suitable for use in a dietary supplement.

In an embodiment, the health product, the cosmetic product or the dietary supplement of any one aspect of the present invention comprises at least one additional excipient selected from a flavoring agent, an antioxidant, a colorant, a mineral, a vitamin, a pH regulator, a protein, a lipid and carrier, and any combination thereof.

In a particularly preferred embodiment, the dietary supplement composition comprises 150 mg of polysaccharide mixture, 2 mg of sodium stearate, and 148 mg of microcrystalline cellulose. In a further particularly preferred embodiment, the dietary supplement composition consists of 150 mg of polysaccharide mixture, 2 mg of sodium stearate, and 148 mg of microcrystalline cellulose.

Another aspect of the present invention is a composition comprising the polysaccharide mixture of the invention for use in increasing the production of SCFA in the gastrointestinal tract, skin, serum or mucosa of a subject.

Another aspect of the present invention is a composition comprising the polysaccharide mixture of the invention for use in improving the function of the microbiota in the gastrointestinal tract, skin, serum or mucosa of a subject.

Another aspect of the present invention is a composition comprising the polysaccharide mixture of the present invention for use in altering the structure of the microbiota in the gastrointestinal tract, skin, serum or mucosa of a subject.

Another aspect of the present invention is a composition comprising the polysaccharide mixture of the invention for use in the treatment of an illness, disease, infection, or ailment suffered by a subject.

Another aspect of the present invention is a composition comprising the polysaccharide mixture of the invention for use in the prevention of an illness, disease, infection, or ailment suffered by a subject.

Another aspect of the present invention is a method for increasing the production of SCFA in the gastrointestinal tract, skin, or mucosa of a subject, comprising the steps of
a) providing a composition comprising a polysaccharide mixture of the present invention,
b) administering an effective amount of the composition of step A) to a subject in need thereof, thereby increasing the production of Short Chain Fatty Acids (SCFA) in the gut, skin, or mucosa microbiota of said subject.

In an embodiment, the method for increasing the production of SCFA in the gastrointestinal tract, skin, or mucosa of a subject comprises increasing the production of SCFA in the gastrointestinal tract, skin, or mucosa of the subject in such way that it is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the method for increasing the production of SCFA in the gastrointestinal tract, skin, or mucosa of a subject comprises increasing the production of SCFA in the gastrointestinal tract, skin, or mucosa of a subject in such a way that it is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

In an embodiment, the method for increasing the production of SCFA in the gastrointestinal tract, skin, or mucosa of a subject comprises altering the structure and/or function of the microbiota in the gastrointestinal tract, skin, or mucosa of the subject in such way that it is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the method for increasing the production of SCFA in the gastrointestinal tract, skin, or mucosa of a subject comprises altering the structure and/or function of the microbiota in the gastrointestinal tract, skin, or mucosa of a subject in such a way that it is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

Another aspect of the present invention is a method for altering the microbiome structure and/or function present in the gastrointestinal tract, skin, or mucosa in a subject, comprising the steps of
a) providing a composition comprising a polysaccharide mixture of the present invention,
b) administering an effective amount of the composition of step A) to a subject in need thereof, thereby altering the microbiome structure and/or function present in the gastrointestinal tract, skin, or mucosa in said subject.

In an embodiment, the method for altering the microbiome structure and/or function present in the gastrointestinal tract, skin, or mucosa in a subject comprises altering the structure and/or function of the microbiota in the gastrointestinal tract, skin, or mucosa of the subject in such way that it is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the method for altering the microbiome structure and/or function present in the gastrointestinal tract, skin, or mucosa of a subject comprises altering the structure and/or function of the microbiota in the gastrointestinal tract, skin, or mucosa of a subject in such a way that it is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

In an embodiment, the method for increasing the production of SCFA in the gastrointestinal tract, skin, or mucosa of a subject, or the method for altering the microbiome structure and/or function present in the gastrointestinal tract, skin, or mucosa in a subject further comprises a customizing step carried out before step a), wherein said customizing step comprises the substeps of
i) taking a stool sample, skin swab or mucosal swab of a subject,
ii) analyzing the genes related to SCFA biosynthesis or polysaccharide degradation in the microbiota by utilizing biomarkers,
iii) formulating a composition comprising a polysaccharide mixture of the present invention based on the analysis of step ii).

### BRIEF DESCRIPTION OF THE FIGURES

FIG. 1 shows anomeric signals from a ¹H ¹³C HSQC 2D Nuclear Magnetic Resonance (NMR) spectrum of Ganoderma lucidum polysaccharide

FIG. 2 shows a ¹H ¹³C HSQC 2D NMR spectrum of Ganoderma lucidum polysaccharide

FIG. 3 shows anomeric signals from a ¹H ¹³C HSQC NMR of Ganoderma sessile polysaccharide

FIG. 4 shows a ¹H ¹³C HSQC NMR of Ganoderma sessile polysaccharide

FIG. 5 shows differences in the microbiome structure of two volunteers, before (visit 1) and after (visit 2) consuming inulin (H), and before and after consuming the mixture of fungal polysaccharides (P). 5A. PCoA Bray Curtis distance. 5B. relative frequencies, species level.

FIG. 6 shows differences in alpha diversity indexes Chao1 (6A) and Shanon (6B) in fecal microbiome before (examination 1) and after (examination 2) consuming inulin (H) and before and after consuming the mixture of fungal polysaccharides (P)

FIG. 7 shows levels of SCFA in an in vitro study before (C0) and after fermentation of inulin (I), G. lucidum polysaccharide (GL) and fungal polysaccharide (GPS) by fecal human microbiota (P1). *** p<0.001, ** p<0.01, *p<0.05

FIG. 8 serum levels of SCFA before (V1) after (V2) 2 weeks of oral intake of inulin (H) at a dose of 3 g/d and fungal polysaccharide (P) at a dose of 150 mg/d.

FIG. 9. Differential expression of endo-beta-1,6-glucanase (B. uniformis) after in vitro fermentation of G. lucidum polysaccharide (GL), inulin (I) and G. sessile polysaccharide (GPS). P1 and P2 are different fecal donors.

### DETAILED DESCRIPTION OF THE INVENTION

Each and every embodiment of the aspects, objects and/or methods of the present invention resulting from the combination of particular embodiments described herein shall be considered as falling within the scope of the present invention.

Any technical terminology used herein shall be understood by the common definition utilized in the art and/or by those skilled in the art, unless otherwise explicitly stated or inferred by context.

As used herein, an “effective amount” is defined as an amount effective, at dosages and for periods of time necessary, to achieve a desired biological result, such as reducing, preventing, or treating an illness, disease, infection or ailment, and/or inducing a particular beneficial effect. The effective amount of a composition or polysaccharide mixture of the present invention may vary according to factors such as age, sex, and weight of the individual.

As used herein, the term “microbiota” refers to the collection of virus, bacteria, archaea and eukarya colonizing any part of the body including, but not limited to, gut, skin and/or mucosa, and any sample that can be obtained from these by knowns methods in the art.

As used herein, the term “microbiome” refers to the genomic, transcriptomic, proteomic, and metabolomic content of the microbiota.

As used herein, the term “microbiome structure” or “structure of microbiome” refers to the collection of bacterial organisms, genomes, transcripts, and proteins, and their corresponding abundancies in the microbiota of a subject, for example, the microbiota that can be found in the gut, skin, mucosa, or samples of any of these.

As used herein, the term “microbiome function” or “function of the microbiome” refers to the physiological effect that the microbiome has on the subject, which may be the result of the functions of the microbiota that make up the microbiome. More particularly, the term refers to the effect that the microbiome has on the body from a health, cosmetic or dietary point of view.

As used herein, the term “dysbiosis” refers to an alteration in the composition and/or function of the microbiota. (adapted from Hrncir et, al 2022)

As used herein, a “prebiotic” is defined as a substrate that is selectively utilized by at least one host microorganism conferring a health benefit. A prebiotic may further comprise a nutritional product and/or ingredient selectively utilized in the microbiome producing health benefits.

As used herein, the term “subject” refers to any vertebrate including, without limitation, humans and other primates (e.g., chimpanzees and other apes and monkey species), farm animals (e.g., cattle, sheep, pigs, goats, and horses), domestic animals (e.g., dogs and cats), laboratory animals (e.g., rodents such as mice, rats, and guinea pigs), and birds (e.g., domestic, wild, and game birds such as chickens, turkeys, and other gallinaceous birds, ducks, geese, and the like). In some embodiments, the subject may be a mammal. In other embodiments, the subject may be a human.

As used herein, the term “health product” refers to any product commonly used for the management of healthcare. The term includes medicines, allied substances, herbs and products derived thereof, and traditional medicines.

As used herein, the term “cosmetic product” refers to any product commonly used for aesthetic purposes and includes any product that can be placed in contact with external parts of the human body or with the mucous membranes of the body.

As used herein, the term “dietary supplement” refers any product or combination of products that are consumed in addition to or as a complement for a diet in order to fulfill a particular need that said diet cannot achieve on its own. The definition includes food supplements, for example, concentrated sources of nutrients (e.g. minerals and vitamins) or other substances with nutritional or physiological effect that are marketed in “dose” form (e.g., pills, tablets, capsules, liquids, or formulations in measured doses).

As used herein, the term “X-Y”, “between X-Y”, “from X to Y” and similar, when referring to a range of values, shall be understood as including values of X and Y.

A first aspect of the present invention is a polysaccharide mixture, wherein said polysaccharide mixture comprises at least one polysaccharide extracted from Ganoderma sessile.

The inventor has found that G. sessile polysaccharides are chemically and functionally different from G. lucidum polysaccharides (see Examples 4 and 5 below). Surprisingly, these structural and functional differences between G. sessile and G. lucidum polysaccharides result in G. sessile polysaccharides showing a clear effect on both function and composition of gut microbiota while G. lucidum polysaccharide does not show significant effects (see Example 6). Furthermore, the inventors found that G. sessile polysaccharides have better prebiotic properties than G. lucidum polysaccharides and homopolysaccharides such as glucans and inulin, as seen in Example 6.

The polysaccharide mixture of the present invention can be used to increase the production of Short Chain Fatty Acids (SCFA) in a microorganism, more specifically in a microorganism that belongs to the gut, skin or mucosa microbiota of a subject.

The polysaccharide mixture of the present invention comprises between 2 and 8 polysaccharides. Preferably the mixture comprises 2, 3, 4, 5, 6, 7 or 8 polysaccharides. More preferably, said mixture comprises 4 polysaccharides.

In an embodiment, the polysaccharide mixture comprises only polysaccharides extracted from Ganoderma sessile.

The polysaccharide mixture of the present invention comprises at least one β-glucan polysaccharide, wherein said β-glucan comprises at least one β glycosidic bond, or both α and β glycosidic bonds, and wherein said glycosidic bonds can be 1-3, 1-4 or 1-6 glycosidic bonds.

In an embodiment, the polysaccharide mixture comprises more than one polysaccharide, wherein at least one of the polysaccharides has a different degree of polymerization from the remaining polysaccharides.

Preferably, the polysaccharide mixture of the present invention comprises polysaccharides having between 4 and 4000 monomers. More preferably between 100 and 3600 monomers.

The polysaccharide mixture comprises at least one polysaccharide comprising at least one type of monosaccharide selected arabinose, xylose, mannose, galactose, glucose, rhamnose, fucose, ribose, fructose. Preferably, the polysaccharide mixture comprises a combination of the aforementioned monosaccharides, more preferably, the polysaccharide mixture comprises arabinose, xylose, mannose, galactose, glucose, fucose and rhamnose.

It should be understood that, when the polysaccharide mixture comprises a combination of the aforementioned monosaccharides, said monosaccharides may be distributed between different polysaccharides within the polysaccharide mixture. For example, and without intent of limiting the scope of the present invention, when the polysaccharide mixture of the present invention comprises glucose, mannose and arabinose, it is to be understood that glucose, mannose and arabinose are present throughout the different polysaccharide molecules that are comprised by the polysaccharide mixture, in any combination.

In a particularly preferred embodiment, the polysaccharide mixture comprises the following monosaccharides expressed in molar percentage: arabinose 14.3-45.1; xylose 14.9-18.1; mannose 12.6-21.8; galactose 12.6-24.6; glucose 7.0-30.0; fucose 1.2-4.6; rhamnose 0.8-1.6.

The polysaccharide mixture of the present invention comprises polysaccharides having a molecular weight between 1 and 650 kDa. In an embodiment, the polysaccharide mixture comprises more than one polysaccharide, wherein at least one of the polysaccharides has a different molecular weight from the remaining polysaccharides.

In a particularly preferred embodiment, the polysaccharide mixture comprises
a polysaccharide with a molecular weight between 1 and 2 kDa, for example 1.26 kDa,
a polysaccharide with a molecular weight between 5 and 10 kDa, for example 9.26 kDa,
a polysaccharide weighing with a molecular weight 50 and 100 kDa, for example 68.03 kDa, and
a polysaccharide weighing with a molecular weight 500 and 1000 kDa, for example 634.38 kDa.

In an embodiment, the polysaccharide mixture comprises polysaccharides with polydispersity index lower than 1.87. Preferably, said polysaccharides have a polydispersity index between 1.00-1.75, more preferably between 1.25 and 1.50.

As seen in Examples 4 and 5, the polysaccharide mixture of G. sessile, comprises different types of monosaccharides, a higher variety of molecular weights and a lower polydispersity index of each individual polysaccharide compared to the polydispersity index of the G. lucidum polysaccharide. These characteristics are associated with an improved prebiotic effect seen in G. sessile polysaccharides.

In a particularly preferred embodiment, the polysaccharide mixture consists of a polysaccharide mixture obtained from Ganoderma sessile.

The fungus used for the production of the polysaccharide mixture of the present invention is Ganoderma sessile. Samples of this fungus can be obtained from collection in natural environments, such as e.g., growing at decaying trees in the United States of America (Loyd et al., 2018). A person skilled in the art may make use of techniques known in the art for the correct identification and purification of the fungus to be used. For example, following the teachings disclosed in Zhou et al., 2015; Loyd A L, 2018; and Loyd et al., 2019.

For example, Ganoderma sessile can be identified by the molecular analysis of the Internal Transcribed Spacer 1 (ITS). For example, for the identification of Ganoderma sessile, techniques are readily available for a person skilled in the art for the identification of this species, (Loyd et al, 2018). An example of a strain of Ganoderma sessile identified in this way correspond to a Ganoderma sessile that contains a ITS1 of SEQ ID NO: 1.

In an embodiment, the polysaccharide mixture comprises polysaccharides extracted from at least one other fungus, wherein said one other fungus comprises at least one fungus from the genus Ganoderma, Hericium, Cordiceps, Inonotus, Lentinus, Trametes, Schizophyllum, Grifola, Tremella, Poria, Flammulina and Pleurotus. More preferably, the one other fungus of the genus Ganoderma.

The polysaccharide mixture of the present invention was obtained by performing a fermentation process, as disclosed herein. Therefore, another aspect of the present invention is a fermentation process of at least one fungus comprising Ganoderma sessile, wherein said fermentation process takes place in a bioreactor of a volume of 10-10000 L, preferably 1-1000 L, more preferably, 1-100 L, and said process comprises concentrating the fermentation product by ultrafiltration.

Preferably, an ultrafiltration membrane of 10 KDa cutoff is employed as an extraction method in the fermentation process to ensure an adequate molecular size of the polysaccharides in the polysaccharide mixture provided by the invention, so that the advantageous effects related to the increase in the production of SCFA can be obtained.

The use of different methods of extraction leads to a higher amount of residual monosaccharides in the mixture as well as salts, and to a loss or dilution of the polysaccharide as an active ingredient. In addition, the presence of other molecules of low molecular size of fungal origin not properly eliminated from the extract can lead to different effects as those intended.

It is understood that, thanks to the teachings provided herein, a person skilled in the art would have the knowledge necessary to adapt the production scale of the polysaccharide mixture of the present invention into a preferred scale, e.g., an industrial scale, pilot scale or laboratory scale, being the main factor the volume at which the fermentation process is performed. As such, the application of the fermentation process in any of these scales shall be considered as falling within the scope of the present invention.

In an embodiment, the ultrafiltration comprises concentrating the fermentation product between 5-15 fold, more preferably the fermentation product is concentrated ten-fold.

The fermentation process of the present invention can be carried out during a period of time ranging from 3-15 days, preferably between 5-10 days, more preferably 5 days.

The fermentation process can be carried out with a temperature ranging from 25-35° C., preferably between 28-32° C., more preferably between 30-32° C.

The fermentation can be performed with a pH ranging from 5.0-6.0, preferably between 5.2-5.7, more preferably between 5.4-5.6.

In particular embodiments, the fermentation can be performed with a percentage of dissolved oxygen of higher than 30%, more preferably a percentage of dissolved oxygen between 15-60%.

The inventor has found that, by employing the aforementioned fermentation conditions including the culture medium composition, pH levels and dissolved oxygen concentration and by and concentrating the fermentation product by ultrafiltration, G. sessile produces a polysaccharide mixture having a composition suitable for the uses described herein as different aspects of the invention.

More specifically, the inventor found that these fermentation conditions are necessary to obtain polysaccharides mixtures having a number average molecular weight (Mn) between 1.00-450 kDa, an average molecular weight (Mw) between 1-650 kDa, and a polydispersity Mw/Mn between 1.25-1.50.

In an embodiment, the fermentation process comprises a step of drying the retentate of the ultrafiltration during a certain amount of time and at certain temperatures. A person skilled in the art would be able to determine the optimal amount of time and temperature to further concentrate the retentate of the process. For example, and without any intent to limit the scope of the present invention, said temperature and time could be 2-48 hours and 40-80° C.

In an embodiment, the fermentation process comprises a pre-culture step prior to the fermentation, wherein an inactive seed culture is incubated in a sunflower seed meal broth, thereby activating it.

In an embodiment, the at least one fungus of the fermentation process comprises Ganoderma sessile. In another embodiment, the fermentation process comprises the fermentation of at least one other fungus, wherein said one other fungus comprises at least one fungus from the genus Ganoderma, Hericium, Cordiceps, Inonotus, Lentinus, Trametes, Schizophyllum, Grifola, Tremella, Poria, Flammulina and Pleurotus. More preferably, the one other fungus is of the genus Ganoderma.

Furthermore, in the state of the art, the most common method for concentrating polysaccharides involves the use of alcohols, specifically ethanol, as described in Yew-Keong et al, 2018 (Extraction and Fractionation of Polysaccharides from a Selected Mushroom Species, Ganoderma lucidum: A Critical Review), however in the fermentation process described herein, the polysaccharides are concentrated by ultrafiltration.

The polysaccharide mixture of the present invention can be used for the manufacture of a composition intended for its administration to a subject. Correspondingly, another aspect of the present invention is a composition, wherein said composition comprises a polysaccharide mixture of the present invention.

In an embodiment, the composition comprises at least 0.1% w/w of the polysaccharide mixture of the present invention. Preferably between 10-95% w/w of the polysaccharide mixture of the present invention, more preferably between 25-75% w/w of the polysaccharide of the present invention, even more preferably 50% w/w of the polysaccharide mixture of the present invention.

In an embodiment, the composition of the present invention can be used to manufacture a dietary supplement, a cosmetic product, or a health product. The daily dose of composition should be enough to achieve an effective amount of polysaccharide delivered. For dietary supplements this minimum was found to be 150 mg, as evidenced in Examples 9 and 10. For cosmetic products, this minimum was found to be 5 mg. For health products for the treatment of diseases or specific health conditions, higher doses may be required, for example 300 mg. The maximum daily dose is limited by the onset of side effects.

When used to manufacture a dietary supplement, a useful daily dose range of the composition of the present invention for human use is from 100-10000 mg, preferably 100-3000 mg, more preferably 300 mg. In another embodiment, the useful daily dose range of the composition is administered in 1-3 servings, preferably 1 serving. In a further embodiment, the administration of the dietary supplement may last 7-60 days, preferably 10-30 days, more preferably 14 days.

When used to manufacture a dietary supplement, a useful daily dose range of the polysaccharide mixture of the present invention for human use comprises 150-3000 mg, preferably 150-600 mg, more preferably 150 mg.

In an embodiment, a dietary supplement of the present invention can be an ingredient for food.

In an embodiment, the dietary supplement comprises at least 10% w/w of the polysaccharide mixture of the present invention. Preferably between 10-95% w/w of the polysaccharide mixture of the present invention, more preferably between 25-75% w/w of the polysaccharide of the present invention, even more preferably 50% w/w of the polysaccharide mixture of the present invention.

When used to manufacture a cosmetic product, a useful daily dose range of the composition of the present invention for human use is from 5-1000 mg, preferably 20-200 mg, more preferably 40 mg. In another embodiment, the useful daily dose range of the composition is administered in 1-3 administrations, preferably 2 administrations. In a further embodiment, the administration of the cosmetic product may last 30-90 days, preferably 45-75 days, more preferably 60 days.

When used to manufacture a cosmetic product, a useful daily dose range of the polysaccharide mixture of the present invention for human use comprises 5-100 mg, preferably 10-50 mg, more preferably 20 mg.

In an embodiment, the cosmetic product comprises at least 0.1% w/w of the polysaccharide mixture of the present invention. Preferably between 0.1-10% w/w of the polysaccharide mixture of the present invention, more preferably between 0.1-1% w/w of the polysaccharide of the present invention, even more preferably 1% w/w of the polysaccharide mixture of the present invention.

When used to manufacture a health product for a person who suffers a disease, illness, infection or ailment, a useful daily dose range of the composition of the present invention for human use is from 0.3-30 g, preferably 0.3-10 g, more preferably 1.5 g. In another embodiment, the useful daily dose range of the composition is administered in 1-4 administrations, preferably 2 administrations. In a further embodiment, the administration of the health product may last 7-120 days, preferably 15-60 days, more preferably 30 days.

When used to manufacture a health product, a useful daily dose range of the polysaccharide mixture of the present invention for human use comprises 150-9000 mg, preferably 150-3000 mg, more preferably 600 mg.

In an embodiment, the health product comprises at least 10% w/w of the polysaccharide mixture of the present invention. Preferably between 10-95% w/w of the polysaccharide mixture of the present invention, more preferably between 25-75% w/w of the polysaccharide of the present invention, even more preferably 50% w/w of the polysaccharide mixture of the present invention.

Depending on the intended use of the composition, as well as on the dosage form thereof, the composition may comprise several additional ingredients. For instance, the composition of the present invention may comprise a flavoring agent, an antioxidant, a colorant, a mineral, a vitamin, a pH regulator, a protein, a lipid and carrier, or any combination thereof.

Preferably, said at least one additional excipient suitable for a dietary supplement is selected from sucrose, herbs flavoring, spices flavoring, fruit flavorings, tocopherols, rosemary extract, ascorbic acid, astaxanthin, phenols, carotenoids, anthocyanins, betacyanins, carotenoids, curcuminoids, Phosphorus, Copper, Potassium, Magnesium, Calcium, Manganese, Iron, Selenium, Zinc, Sulfur, B vitamins (B2, B3, folate, B5, B12), Vitamin C, Vitamin D, Vitamin E, citric acid, sodium carbonate, sodium bicarbonate, sodium citrate, oleic acid, linoleic acid, linolenic acid, crystalline cellulose, carboxymethylcellulose, dextrin, starch, polydextrose, and at least one plant or mushroom protein.

In an embodiment, the health product comprises at least one additional excipient suitable for a health product selected from sucrose, herbs flavoring, spices flavoring, fruit flavorings, tocopherols, rosemary extract, ascorbic acid, astaxanthin, phenols, carotenoids, anthocyanins, betacyanins, carotenoids, curcuminoids, Phosphorus, Copper, Potassium, Magnesium, Calcium, Manganese, Iron, Selenium, Zinc, Sulfur, B vitamins (B2, B3, folate, B5, B12), Vitamin C, Vitamin D, Vitamin E, citric acid, sodium carbonate, sodium bicarbonate, sodium citrate, oleic acid, linoleic acid, linolenic acid, crystalline cellulose, carboxymethylcellulose, dextrin, starch, polydextrose, and at least one plant or mushroom protein.

In an embodiment, the cosmetic product comprises at least one additional excipient suitable for a cosmetic product selected from sucrose, herbs flavoring, spices flavoring, fruit flavorings, tocopherols, rosemary extract, ascorbic acid, astaxanthin, phenols, carotenoids, anthocyanins, betacyanins, carotenoids, curcuminoids, Phosphorus, Copper, Potassium, Magnesium, Calcium, Manganese, Iron, Selenium, Zinc, Sulfur, B vitamins (B2, B3, folate, B5, B12), Vitamin C, Vitamin D, Vitamin E, citric acid, sodium carbonate, sodium bicarbonate, sodium citrate, oleic acid, linoleic acid, linolenic acid, crystalline cellulose, carboxymethylcellulose, dextrin, starch, polydextrose, and at least one plant or mushroom protein.

In an embodiment, the composition of the present invention may comprise at least one flavoring agent selected from sucrose, herbs, spices, fruits and other natural flavorings.

In an embodiment, the composition of the present invention may comprise at least one antioxidant selected from tocopherols, rosemary extract, ascorbic acid, astaxanthin, phenols, and carotenoids.

In an embodiment, the composition of the present invention may comprise at least one colorant selected from anthocyanins, betacyanins, carotenoids, and curcuminoids.

In an embodiment, the composition of the present invention may comprise at least one mineral selected from Phosphorus, Copper, Potassium, Magnesium, Calcium, Manganese, Iron, Selenium, Zinc, Sulfur.

In an embodiment, the composition of the present invention may comprise at least one vitamin selected from a B vitamin (vitamin B2, vitamin B3, folate, vitamin B5, vitamin B12), Vitamin C, Vitamin D, and Vitamin E.

In an embodiment, the composition of the present invention may comprise at least one pH regulator selected from citric acid, sodium carbonate, sodium bicarbonate, sodium citrate.

In an embodiment, the composition of the present invention may comprise at least one protein, more preferably at least one mushroom or plant protein.

In an embodiment, the composition of the present invention may comprise at least one lipid selected from oleic acid, linoleic acid, and linolenic acid.

In an embodiment, the composition of the present invention may comprise at least one carrier selected from crystalline cellulose, carboxymethylcellulose, dextrin, starch, polydextrose.

In a particularly preferred embodiment, the composition of the present invention comprises moisture 10%; ash; lipids, proteins, carbohydrates, minerals Cu, Fe, Mn, Zn, K, Mg, Ca, P, S, total glucan 10.5%.

The composition of the present invention may be suitable for oral, rectal, nasal, topical (including buccal and sub-lingual), or vaginal administration. In an embodiment, the composition of the present invention is administered via oral, rectal, nasal, topical (including buccal and sub-lingual), or vaginal administration.

Preferably, when the composition is used to manufacture a dietary supplement, the administration is oral administration, and when the composition is a cosmetic product the administration is via topical administration.

Another aspect of the present invention is a composition comprising the polysaccharide mixture of the invention for use in increasing the production of SCFA in the gastrointestinal tract, skin, serum or mucosa of a subject.

In an embodiment, the composition for use in increasing the production of SCFA further comprises at least one additional excipient selected from a flavoring agent, an antioxidant, a colorant, a mineral, a vitamin, a pH regulator, a protein, a lipid, and a carrier, and any combination thereof.

In an embodiment, the composition for use in increasing the production of SCFA is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the composition for use in increasing the production of SCFA is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

In an embodiment, the composition for use in increasing the production of SCFA is useful for improving the function of the microbiota in the gastrointestinal tract, skin, serum or mucosa of a subject, or for altering the structure of the microbiota in the gastrointestinal tract, skin, serum or mucosa of a subject.

Another aspect of the present invention is a composition comprising the polysaccharide mixture of the invention for use in improving the function of the microbiota in the gastrointestinal tract, skin, serum or mucosa of a subject.

In an embodiment, the composition for use in improving the function of the microbiota further comprises at least one additional excipient selected from a flavoring agent, an antioxidant, a colorant, a mineral, a vitamin, a pH regulator, a protein, a lipid, and a carrier, and any combination thereof.

In an embodiment, the composition for use in improving the function of the microbiota is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the composition for use in improving the function of the microbiota is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

Another aspect of the present invention is a composition comprising the polysaccharide mixture of the present invention for use in altering the structure of the microbiota in the gastrointestinal tract, skin, serum or mucosa of a subject.

In an embodiment, the composition for use in altering the structure of the microbiota further comprises at least one additional excipient selected from a flavoring agent, an antioxidant, a colorant, a mineral, a vitamin, a pH regulator, a protein, a lipid, and a carrier, and any combination thereof.

In an embodiment, the composition for use in altering the structure of the microbiota is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In an embodiment, the composition for use in altering the structure of the microbiota is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

Another aspect of the present invention is a composition comprising the polysaccharide mixture of the invention for use in the treatment of an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In a particular embodiment, the composition for use in the treatment of an illness, disease, infection, or ailment is for use in the treatment of gut disbyosis. Preferably, wherein the composition comprises a 150-3000 mg, preferably 150-600 mg, more preferably 150 mg of a polysaccharide mixture of the present invention.

In an embodiment, the composition for use in the treatment of an illness, disease, infection, or ailment suffered by a subject further comprises at least one additional excipient selected from a flavoring agent, an antioxidant, a colorant, a mineral, a vitamin, a pH regulator, a protein, a lipid, and a carrier, and any combination thereof.

Another aspect of the present invention is a composition comprising the polysaccharide mixture of the invention for use in the prevention of an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In a particular embodiment, the composition for use in the prevention of an illness, disease, infection, or ailment is for use in the prevention of gut dysbiosis. Preferably, wherein the composition comprises a 150-3000 mg, preferably 150-600 mg, more preferably 150 mg of a polysaccharide mixture of the present invention.

In an embodiment, the composition for use in the prevention of an illness, disease, infection, or ailment suffered by a subject further comprises at least one additional excipient selected from a flavoring agent, an antioxidant, a colorant, a mineral, a vitamin, a pH regulator, a protein, a lipid, and a carrier, and any combination thereof.

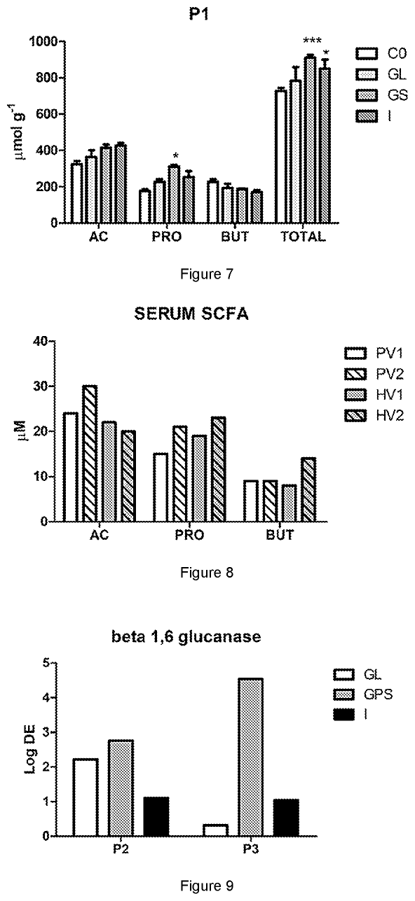

The inventor has found that there are individuals that respond better to G. sessile polysaccharide when compared to inulin. This benefit involves a higher production of SCFA, especially acetate and propionate (FIG. 7). Inulin is a fructan polysaccharide with globally recognized prebiotic properties usually used for increasing the number of beneficial bacteria in the gut, such as Bifidobacterium spp, and Lactobacillus spp.

Thus, another aspect of the present invention is a method for increasing the production of SCFA in the gastrointestinal tract, skin, or mucosa of a subject, wherein said method comprises the steps of
a) providing a composition comprising a polysaccharide mixture of the present invention,
b) administering an effective amount of the composition of step a) to a subject in need thereof, thereby increasing the production of Short Chain Fatty Acids (SCFA) in the gut, skin or mucosal microbiota of said subject.

The method of increasing the production of SCFA in the gastrointestinal tract, skin, or mucosa of a subject of the present invention can be utilized to treat or prevent an illness, disease, infection, or ailment suffered by a subject, wherein the illness, disease, infection, or ailment can be treated or prevented by producing an increasing in the production of SCFA of the microbiota.

In an embodiment, the method of increasing the production of SCFA in the gastrointestinal tract, skin, or mucosa of a subject of the present invention can be utilized to treat or prevent an illness, disease, infection, or ailment that takes place in the gastrointestinal tract, skin or mucosa, or distant organs.

In an embodiment, said illness, disease, infection or ailment includes, but is not limited to, any one of gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, malnutrition, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, immunosuppression, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases or ailments such as polycystic ovarian syndrome, cancer, pregnancy, menopause, reproductive dysfunction, endometriosis, diseases related to the brain-gut-axis such as autism, depression, anxiety, and schizophrenia.

Alternatively, the method of increasing the production of SCFA in the gastrointestinal tract, skin, or mucosa of a subject of the present invention can be utilized for nutritional and/or cosmetic purposes, for example, losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

The method of increasing the production of SCFA in the gastrointestinal tract, skin, or mucosa of a subject of the present invention comprises increasing the production of at least one SCFA. Preferably, the method of increasing the production of SCFA in the gastrointestinal tract, skin, or mucosa of a subject comprises increasing the production of at least one SCFA. In a preferred embodiment said SCFA is selected from acetate, propionate, and butyrate.

Furthermore, the method of increasing the production of SCFA in the gastrointestinal tract, skin, or mucosa of a subject of the present invention comprises increasing the production of SCFA in at least one microorganism of the gut, skin or mucosal microbiota of the subject. In a preferred embodiment, said at least one microorganism is selected from Akkermansia spp., Anaerobutyricum spp., Alistipes spp., Anaerostipes spp., Bacteroides spp., Bacteroides vulgatus, B. caccae, B. uniformis, B. thetaiotaomicron, Bifidobacterium spp., Bifidobacterium adolescents, B. animalis, B. breve, B. bifidum, B. longum, B. angulatum, Blautia spp., Clostridium spp., Coprococcus spp., Eubacterium spp., Faecalibacterium spp., Faecalibacterium prausnitzii, Lachnospira eligens, Lactobacillus spp., Phocaeicola spp., Phocaeicola vulgatus, P. dorei, Prevotella spp., Roseburia spp., Ruminococcus spp., Streptococcus spp., Subdoligranulum spp., and Veillonella spp.

The method of increasing the production of SCFA in the gastrointestinal tract, skin, or mucosa of a subject of the present invention comprises administering the composition of the present invention by any suitable route, including, but not limited to, oral, rectal, nasal, topical (including buccal and sub-lingual), or vaginal administration.

Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or administration, or in multiple servings or administrations spaced throughout the day.

In an embodiment, the method for increasing the production of SCFA in the gastrointestinal tract, skin, or mucosa of a subject comprises altering the structure and/or function of the microbiota in the gastrointestinal tract, skin, or mucosa of the subject. Preferably, in such way that it is useful for treating or preventing an illness, disease, infection, or ailment suffered by a subject. Preferably, wherein said illness, disease, infection, or ailment is selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

In another embodiment, the method for increasing the production of SCFA in the gastrointestinal tract, skin, or mucosa of a subject comprises altering the structure and/or function of the microbiota in the gastrointestinal tract, skin, or mucosa of a subject in such a way that it is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

As will be understood by those skilled in the art, servings or administrations need not be limited to daily servings or administrations, and may be performed between every three hours to every third day or other convenient effective basis. The serving or administration on a given day may be in a single serving or administration or in multiple servings or administrations spaced throughout the day depending on the exigencies of the situation. Likewise, servings or administrations maybe perform several times throughout the day, and may be performed every regular number of hours, for example, every 3, 4, 6, or 8 hours. The exact number of servings or administrations depends on the method of application.

To show an effect in the production of SCFA, these administrations must ensure a constant supply of the polysaccharide mixture of the present invention to the gut, skin or mucosa for a minimum period of at least one week. It should be noted that the polysaccharide mixture reaches the colon depending on the transit time, which may differ depending on the health status or disease of the subject.

In an embodiment, the step b) comprises administering the composition in a useful daily dose range from 0.15-10 g, preferably 0.15-5 g, more preferably 0.9 g. In another embodiment, the step b) comprises administering the composition in a useful daily dose range from 1-4 administrations, preferably 2 administrations. In a further embodiment, the step b) comprises administering the composition in a useful daily dose range for 7-120 days, preferably 15-60 days, more preferably 30 days.

The inventor has surprisingly found that the administration of the composition comprising the mixtures of polysaccharides of the present invention generates a reduction in the abundance of potentially pathogenic bacteria, favoring the predominance of Bacteroides spp. when compared to inulin as reference in vitro. Additionally, the inventor compared and concluded that there are differences between the SCFA production and polysaccharide degrading enzymes between polysaccharides produced by G. sessile and G. lucidum, as it can be appreciated in Examples 4 and 5.

Therefore, another aspect of the present invention is to provide a method for altering the microbiome structure and/or function present in the gastrointestinal tract, skin, or mucosa of a subject, wherein said method comprises the steps of
a) providing a composition comprising a polysaccharide mixture of the present invention,
b) administering an effective amount of the composition of step a) to a subject in need thereof, thereby altering the microbiome structure present in the gastrointestinal tract, skin, or mucosa in said subject.

The method of altering the microbiome structure and/or function comprises increasing the total and/or relative number of at least one target microorganism, wherein said at least one microorganism is selected from Akkermansia spp., Anaerobutyricum spp., Alistipes spp., Anaerostipes spp., Bacteroides spp., Bacteroides vulgatus, B. caccae, B. uniformis, B. thetaiotaomicron, Bifidobacterium spp., Bifidobacterium adolescents, B. animalis, B. breve, B. bifidum, B. longum, B. angulatum, Blautia spp., Clostridium spp., Coprococcus spp., Eubacterium spp., Faecalibacterium spp., Faecalibacterium prausnitzii, Lachnospira eligens, Lactobacillus spp., Phocaeicola spp., Phocaeicola vulgatus, P. dorei, Prevotella spp., Roseburia spp., Ruminococcus spp., Streptococcus spp., Subdoligranulum spp., and Veillonella spp.

The same considerations provided for the method of increasing the production of SCFA in the gastrointestinal tract, skin, or mucosa of a subject described above apply to the method of altering the microbiome structure and/or function in the gastrointestinal tract, skin, or mucosa of a subject as well.

In an embodiment, the method of increasing the production of SCFA in the gastrointestinal tract, skin, or mucosa of a subject or the method of altering the microbiome structure and/or function present in the gastrointestinal tract, skin, or mucosa in a subject the further comprise a customizing step carried out before step a), wherein said customizing step comprises the substeps of
i) taking a stool sample, skin swab or mucosal swab of the subject,
ii) analyzing the genes related to SCFA biosynthesis or polysaccharide degradation in the fecal microbiota by utilizing biomarkers,
iii) formulating a composition comprising a polysaccharide mixture of the present invention based on the analysis of step II).

In an embodiment, the substep II) of the customizing step comprises analyzing at least one gene selected from pyruvate dehydrogenase, phosphate acetyl transferase, acetate kinase, pyruvate ferredoxin oxidoreductase, acetate CoA-ligase, pyruvate formate-lyase, succinylCoA: acetate CoA transferase, propionyl-CoA: succinate CoA transferase, lactate CoA transferase, lactoyl-CoA dehydratase, acryloyl-CoA reductase, propanoate-CoA transferase, propanediol dehydratase, CoA dependent propionaldehyde dehydratase, phosphate propanoyltransferase, propionate kinase, acetyl-CoA acetyltransferase, 3-hydroxybutyryl-CoA dehydrogenase, 3-hydroxybutyryl-CoA dehydratase, butanoyl-CoA dehydrogenase (NAD+, ferredoxin), phosphotransbutyrylase, butyrase kinase, butanoyl-CoA: acetate CoA-transferase, 4-hydroxybutyrate CoA transferase, 4-hydroxybutyrate dehydrogenase, succinate semialdehyde dehydrogenase, succinyl-CoA: CoA transferase, endoglucanase/cellulase, glucan endo-1,3-β-D-glucosidase (endo-1,3-β-glucanase), glucan 1,3-β-glucosidase (1,3-β-glucan glucohydrolase), glucan endo-1,2-β-glucosidase (endo-1,2-β-glucanase), licheninase (1,4-β-glucan endohydrolase), glucan 1,4-β-glucosidase (β-1,4-β-glucanase), glucan endo-1,6-β-glucosidase (endo-1,6-β-glucanase), cellulose 1,4-β-cellobiosidase (non-reducing end) (1,4-β-glucan cellobiosidase), endoxylanase, xylosidase, xylanase, pectin lyase, beta-fructofuranosidase, galacturonidase, rhamnogalacturonan endolyase, rhamnogalacturonan exolyase, galacturonyl hydrolase, arabinofuranosidase, mannanase, chitinase, and endochitinase.

In an embodiment, the substep II) of the customizing step comprises identifying the presence or absence of the biomarkers, or quantifying their abundances in a stool sample, or skin or mucosal swab of the subject, and predict through an algorithm the response to a polysaccharide.

Genetical analysis method for identifying the presence or absence of biomarkers, or quantifying them are within the knowledge of a person skilled in the art and include, but are not limited to, gel electrophoresis, PCR, qPRC, RT-PCR, qPCR, Next generation Sequencing, transcriptomics, proteomics, among others.

In an embodiment, the substep II) of the customizing step comprises utilizing at least one biomarker selected from beta-fructofuranosidase of Roseburia inulinivorans, acetate kinase of Lactobacillus spp., butyrate kinase of Coprococcus eutactus, propanoate CoA transferase of Coprococcus catus, endo-β-1,6-glucanase of Bacteroides vulgatus.

The use of biomarkers can be performed by any biomarking technique known in the state of the art and falling within the knowledge of a person skill in the art. Methods for using biomarkers thus include, but are not limited to PCR, qPCR, NGS, microarray, Real Time PCR, RT-qPCR, hybridization, gel electrophoresis, mass spectrometry.

In an embodiment, the substep III) of the customizing step comprises modifying the polysaccharide mixture based on the results of the substep II). As an example, and without intent to limit the scope of the present invention, when biomarkers of first degrading enzymes of at least one specific polysaccharide are detected in the stool sample, or skin swab or mucosal swab of the subject, and biomarkers of enzymes related to SCFA biosynthesis belonging to first and/or second degraders of said at least one specific polysaccharide are detected in the same sample, a composition containing that said at least one specific polysaccharide is used for increasing the production of SCFA in the gastrointestinal tract, skin or mucosa of the subject.

### EXAMPLES

The following examples shall be understood only as exemplary embodiments of the different aspects, objects and/or methods of the present invention and are not intended to limit the scope of said invention in any way.

### Example 1: Exemplary Embodiment of a Ganoderma sessile Strain

Details on the Ganoderma sessile strain used in the following examples are provided below.

Methods for identification of Ganoderma sessile are available in the prior art, such as the one disclosed in Viceconte et al, 2021. (Ganoderma sessile is a fast polysaccharide producer among Ganoderma species). For further identification of Ganoderma sessile strains, methods are disclosed in the document The Ganoderma weberianum-resinaceum lineage: multilocus phylogenetic analysis and morphology confirm G. mexicanum and G. parvulum in the Neotropics, MycoKeys, 59, 95-131, 2019 [DOI: 10.3897/mycokeys.59.33182]
Name: Ganoderma sessile Murril
Collection Accession number: MUCL 38061
Collection: Belgian Coordinated Collection of Microorganisms (BCCM)
Misapplied names: Ganoderma lucidum (W. Curtis: Fries) Karsten; Ganoderma sessile Murrill
Organism type: Filamentous
Other collections' numbers: FCUP 628
History: 1988, D. Zuch->T. H. Quimio (FCUP)->1993, MUCL
Conditions for growth: MUCL medium MA2, 20C
Origin: America, United States
Literature: Cabarroi-Hernandez Milay, Villalobos-Arámbula Alma Rosa, Torres-Torres Mabel Gisela, Decock Cony, Guzmán-Dávalos Laura, The Ganoderma weberianum-resinaceum lineage: multilocus phylogenetic analysis and morphology confirm G. mexicanum and G. parvulum in the Neotropics, MycoKeys, 59, 95-131, 2019 [DOI: 10.3897/mycokeys.59.33182]

### Example 2: In Vitro Assay—General Experimental Procedure

Stool samples were collected from three healthy volunteers. A protocol for recruitment of volunteers was previously approved by the Ethics Committee of Hospital Italiano Regional del Sur, Bahia Blanca, Argentina, accredited by the Minister of Health of Buenos Aires Province under No. 020/2010, page 55, Act book No. 1, dated Sep. 13, 2019. Expiration of accreditation 7 Dec. 2025.

### —Inclusion Criteria

1. Age ≥18 and <66
2. Normal weight and overweight (18.5≤BMI <30)

### —Exclusion Criteria

1. Obesity: BMI ≥30
2. Low weight: BMI <18.5
3. History of chronic or autoimmune disease.
4. History of gastrointestinal disorders.
5. Acute illness in the last week.
6. Antibiotic therapy in the last three months.
7. Hospitalization in the last three months.
8. Vegans, vegetarians, people on special diets or consumption of dietary fiber.
9. Pregnancy and breastfeeding

The in vitro fermentation assays on stool samples were performed in the basal medium described by Long et al. (2015) modified as follows:
casein peptone 2 g L⁻¹, yeast extract 2 g L⁻¹, NaHCO₃ 2 g L⁻¹, L-cysteine 0.05% w/v, NaCl 0.1 g L⁻¹, K₂HPO₄ 40 mg L⁻¹, KH₂PO₄ 40 mg L⁻¹, MgSO₄·7H₂O 10 mg L⁻¹, CaCl₂)·6H₂O 10 mg L⁻¹, vitamin K 10 μL L⁻¹, Tween 80 2 mL L⁻¹ and hemin 5 mg L⁻¹, adjusted to pH 7.4 and sterilized at 121° C., 1 atm, for 15 min.

Stool was suspended in the modified basal medium to a final concentration of 10% w/v. Polysaccharides were dissolved in the said medium and added to reach a final concentration of 10 mg/mL (1% w/v). Polysaccharides were fermented at 37° C. for 6 hours in an anaerobic jar containing an anaerobic gas generator bag. Controls before and after fermentation contained equal volumes of medium without polysaccharides. Controls without stool were also included to test sterility. Total DNA and RNA before and after fermentation were extracted for further analysis of the microbial composition and gene expression by qPCR.

SCFAs were assessed in supernatants by Gas Chromatography-Mass Spectrometry (GC-MS). The effects of polysaccharides of G. sessile on the fecal microbiota function were compared with the effects of G. lucidum β-glucans and inulin in the same conditions. Each assay was run in triplicate.

The following is the description of the preparation and method as described in Long, W., Xue, Z., Zhang, Q. et al. (2015). https://doi.org/10.1038/srep13469

### Preparation of BCM Batch Culture Systems (Long et al; 2015)

The BCM medium contained peptone at 2 g 1⁻¹, yeast extract at 2 g 1⁻¹, NaCl at 0.1 g 1⁻¹, K₂HPO₄ at 40 mg 1⁻¹, KH₂PO₄ at 40 mg 1⁻¹, MgSO₄·7H₂O at 10 mg 1⁻¹, CaCl₂)·6H₂O at 10 mg 1⁻¹, NaHCO₃ at 2 g 1⁻¹, L-cysteine at 0.05%, vitamin K at 10 μl 1⁻¹, Tween 80 at 2 ml 1⁻¹ and hemin at 5 mg 1⁻¹, adjusted to pH 7.4.

A fresh fecal sample was collected from a healthy woman, age 27, who had no known metabolic or gastrointestinal diseases and had taken no antibiotics or prebiotics for three months prior to the study. Written informed consent was obtained from this donor. The 10% (w/v) fecal slurry was prepared by diluting the fecal sample in sterile modified BCM medium, thoroughly suspended, and placed into an anaerobic jar at 37° C. within 30 min after collection.

The total volume of each culture system was 5 ml. A fecal slurry was prepared with fresh stool and BCM medium at a final concentration of 10% w/v. Negative control cultures consisted of culture medium and inoculum but no prebiotic substrate. Prebiotic cultures consisted of culture medium, inoculum, and a prebiotic (1%, w/v), which was Ganoderma lucidum polysaccharide, G. sessile polysaccharide or inulin. Cultures of negative control and prebiotic treatments were incubated in an anaerobic jar at 37° C. without stirring, and samples were dynamically collected at 0 and 6 h for both prebiotic and negative control cultures.

### Example 3: Obtaining Ganoderma sessile Polysaccharides Via Fermentation in a Bioreactor

Stock cultures were preserved in Malt extract-Yeast extract-Glucose (MYG) agar at −70° C., with 10% v/v glycerol as cryoprotectant. Active cultures were preserved at 4° C. in slant cultures with MYG agar. Composition of MYG agar per liter was: yeast extract 2 g, malt extract 20 g, dextrose 10 g, agar 20 g, pH=6.0.

Seed culture. Petri dishes containing 20 mL of MYG agar were inoculated with active cultures and incubated for 5 days at 30±0.1° C.

Pre-culture. 2 L Erlenmeyers containing production medium were inoculated with 8-mm-diameter agar plugs of seed cultures, 12 plugs each 100 mL media. The production medium was prepared by adding 1 volume of sunflower seed meal broth to 3 volumes of MYG broth followed by sterilization at 121° C.-1 atm, 30 min. The sunflower seed meal broth was obtained by suspending 6% m/v sunflower seed meal in water and autoclaving at 121° C.-1 atm, 30 min. Cultures were incubated for 5 days at 30±0.1° C.

Bioreactor. Fermentation was conducted in a Bioflo® 5000 New Brunswick bioreactor, with a 29.53×59.06 cm 40 L vessel, equipped with six-bladed 316 L stainless steel, Rushton blade impellers of 12.38 cm diameter, at 1 vvm and 30±1° C., 1% v/v inoculum for 5 days. The pH was maintained at 5.5±0.1 by pumping a 1M NaOH solution. To keep the dissolved oxygen above 50%, agitation was held at 139 rpm in the first, second and third day of fermentation, and increased and maintained at 233 rpm in day 4 and 5 until harvesting. In the third day a 30 min pulse of 584 rpm was applied.

Downstream. The culture was passed through a muslin cylindrical filter to remove the biomass. The process was repeated until observing less than 5 hyphae or cells per field under light microscope. The filtrate was ten-fold concentrated by ultrafiltration using a 10 kDa Pellicon P2C010C05 (Ultracel®) cassette. The retentate was dried for 72 hours at 60° C. yielding 1.92 g of polysaccharide per liter of culture.

It was found that the ultrafiltration step is essential to obtain the adequate molecular size of the polysaccharide mixture for the obtention of effects for the different uses described herein. It was found that a different method of extraction leads to a higher amount of monosaccharides and salts in the mixture, as well as loss or dilution of the polysaccharide as an active ingredient. In addition, the presence of other molecules of fungal origin having a low molecular size not properly eliminated from the extract will lead to different effects on the subject.

### Example 4: Chemical Characterization of Ganoderma sessile Polysaccharide

The crude polysaccharide extract was dissolved in water and deproteinized by adding an equal volume of 25% w/v trichloroacetic acid and incubating at 4° C. for 1 h. Proteins were removed by centrifugation. The resulting supernatant was neutralized and dialyzed against tap water for 48 h and against distilled water for 24 h in a dialysis tubing cellulose membrane with a molecular cut-off of 6-8 kDa, 7.9 ml/cm, centrifuged at 11000 rpm, 10° C. for 30 min and further lyophilized.

Monosaccharide composition in molar percentage:
arabinose 44.7±0.4;
xylose 15.5±0.6;
mannose 14.7±2.1;
galactose 13.6±1.0;
glucose 8.5±1.5;
fucose 1.9±0.7;
rhamnose 1.2±0.4.

The polysaccharide of Ganoderma sessile consists of four polysaccharides with the following molecular weight distribution:

| Peak | Number | Weight | |
|---|---|---|---|
| number | Average | Average | |
| in order | Molecular Weight | Molecular Weight | Polydispersity |
| of elution | (Mn) in kDa | (Mw) in kDa | Mw/Mn |
| 1 | 487.89 | 634.38 | 1.30 |
| 2 | 51.16 | 68.03 | 1.33 |
| 3 | 6.19 | 9.26 | 1.50 |
| 4 | 1.00 | 1.26 | 1.26 |

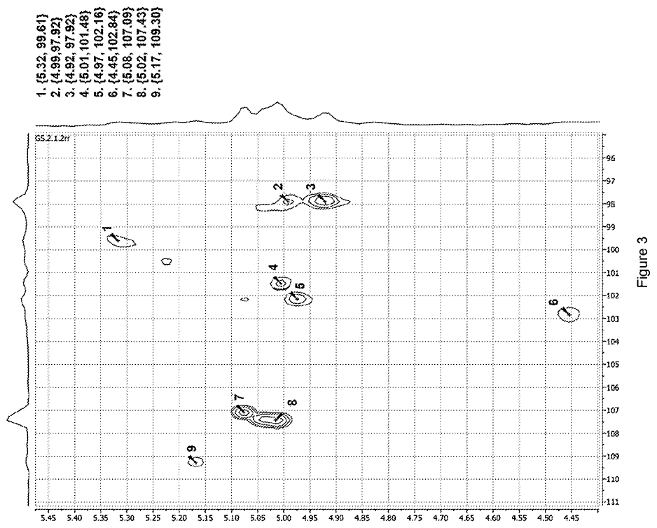

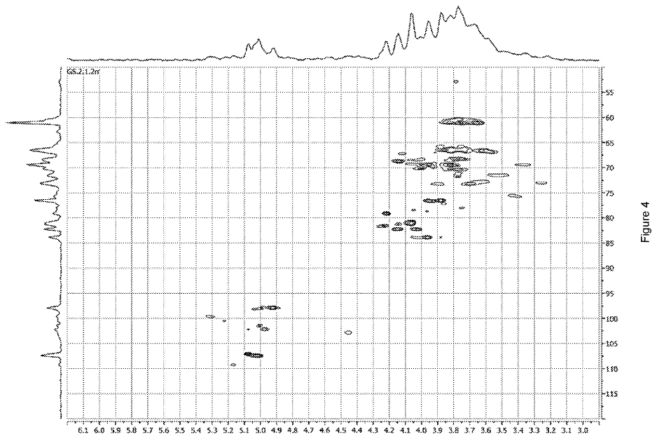

Further characterization of said polysaccharides can be found in FIGS. 3 and 4 herein. The graphs of said Figures have the following Signal assignments: 5/97.9; 4.97/102.1; 5/101.5 ppm α-D-mannose and α-D-galactose. 109.3/5.17; 107.1/5.08; 107.4/5.02 ppm non reducing α-L-arabinofuranoses. NMR studies confirm that this mixture of polysaccharides contains both α and β glucans, as observed in the polysaccharide of G. lucidum. However, according to these studies the most abundant polysaccharide of G. sessile shows an α-L-arabinofuranose backbone.

### Example 5: Chemical Characterization of Ganoderma lucidum Polysaccharide

An amount of 4 g of commercial product (8 caps containing 500 mg each of Reishi mushroom standardized extract, 10% polysaccharides) were boiled in 100 mL purified water for 1 h. After cooling to room temperature, 4 volumes of 96° ethanol were added. The mixture was centrifuged at 650×g for 10 min, the supernatant was removed, and the crude precipitated polysaccharides were dried at 60° C. for 2 h.

The crude polysaccharide extract was dissolved in water and deproteinized by adding an equal volume of 25% w/v trichloroacetic acid and incubating at 4° C. for 1 h. Proteins were removed by centrifugation. The resulting supernatant was neutralized and dialyzed against tap water for 48 h and against distilled water for 24 h in a dialysis tubing cellulose membrane with a molecular cut-off of 6-8 kDa, 7.9 ml/cm, centrifuged at 11000 rpm, 10° C. for 30 min and the supernatants were further lyophilized.

Monosaccharide composition in molar percentage:
glucose 97.5±1.8,
arabinose 0.9±0.3;
galactose 0.8±0.8;
xylose 0.5±0.2.

The purified polysaccharide consisted of one polysaccharide with the following characteristics:

| Peak | Number Average | Weight Average | |
|---|---|---|---|
| number | Molecular | Molecular | |
| in order | Weight | Weight | Polydispersity |
| of elution | (Mn) in kDa | (Mw) in kDa | Mw/Mn |
| 1 | 7.51 | 14.04 | 1.87 |

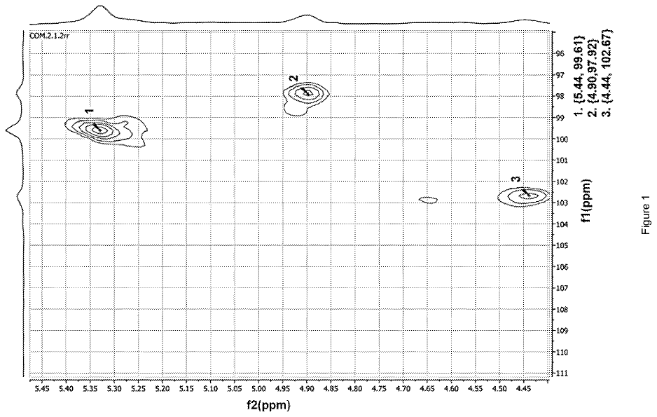

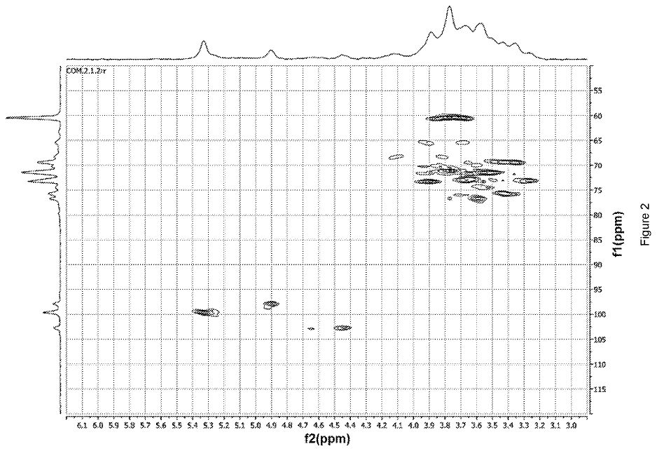

Further characterization of said polysaccharides can be found in FIGS. 1 and 2 herein. The graphs of said Figures have the following Signal assignments: 99.6/5.33 ppm α-D-glucose. 97.9/4.90 ppm (1-6) α-D-glucose. 102.7/4.44 ppm β-D-glucose. NMR studies show the presence of both α and β glucose bonds with 1-6 glycosidic linkages.

### Example 6: Comparative In Vitro Results for G. lucidum, G. Sessile Polysaccharides and Inulin

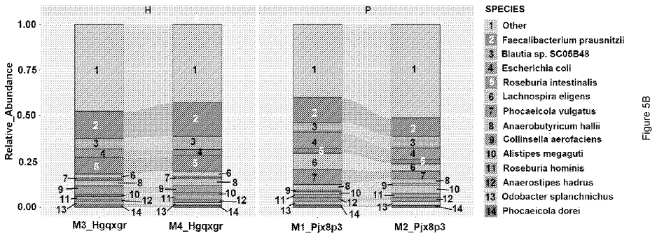

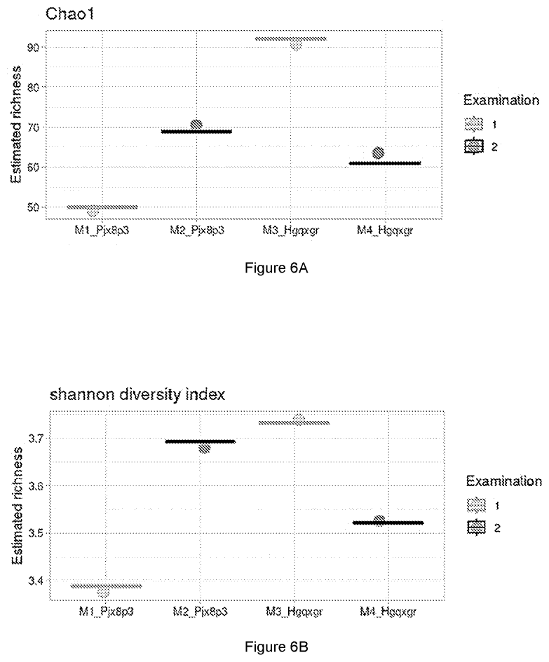

In general, fecal microbiota was not responsive to Ganoderma lucidum polysaccharide regarding production of SCFA in vitro. While both Ganoderma sessile polysaccharide and inulin increased total SCFA depending on person, Ganoderma sessile polysaccharide increased preferentially acetate and/or propionate depending on person (FIG. 6).

The fecal microbiome is differently affected by inulin and Ganoderma sessile polysaccharide treatments. The most abundant species after G. sessile polysaccharide treatment was Phocaeicola vulgatus, while for most participants, the most abundant species after inulin treatment was Bifidobacterium adolescentis.

In most participants the polysaccharide of G. sessile reduced the abundance of potentially pathogenic species such as Clostridioides difficile when compared to both inulin and G. lucidum polysaccharides treatments.

### Example 7: Formulation of a Culture Media for the Production of Polysaccharides in Ganoderma sessile

An example of a composition for the production of polysaccharides for use in Ganoderma sessile is described as follows.

A preferred culture media for Ganoderma sessile comprises
A) a source of carbon (20 g/L), wherein said source of carbon can be glucose, sucrose, or maltose, in any one of the following combinations:
   i) glucose only
   ii) glucose+sucrose (in a 1:2 relationship)
   ii) glucose+maltose (in a 1:2 relationship)
B) a source of nitrogen, wherein said source of nitrogen can be NH₄Cl (12-18 mg/L) or yeast extract (2 g/L) comprising:
   i) up to 35% of protein
   ii) up to 0.5% of fat, and optionally comprises
C) KH₂PO₄ 0.5% w/w, or
D) MgSO₄ 0.2% w/w, or
E) Vitamin B complex.

### Example 8: DNA and RNA Analysis Via qPCR and Rt-qPCR

DNA was extracted with the kit QIAamp PowerFecal Pro DNA Kit (QIAGEN) following the instructions of the supplier. Bacterial genomes were sequenced by Oxford Nanopore Technologies.

RNA was extracted with the ARN-PrepZOL HIGHWAY (INBIO HIGHWAY), following the protocol of the supplier. cDNA was synthesized with the Reverse Transcriptase Kit M-MLV kit (INBIO HIGHWAY), following the instructions of the supplier, and amplified with the Master Mix QuantiNova™ SYBR® Green PCR Kit (QIAGEN) in a Mic qPCR Cycler, in the following conditions: 10 μL vol, 2× SYBR® Green PCR Master Mix 1×, Primer Fw 700 nM, Primer Rv 700 nM, cDNA 100 ng/reaction, activation for 2 min at 95° C., followed by 60 amplification cycles of 5 sec at 95° C., 10 sec at 55° C. and 10 sec at 60° C. Samples were quantified with a DeNovix spectrophotometer. The melting curves were performed increasing temperature by 0.3° C. per cycle starting at 72 to 95° C. ct values of each target gene were normalized to ct values of the 16S rRNA gene in each sample and compared to normalized values of controls before fermentation.

As shown in FIG. 9 the differential expression of the 1,6 betaglucanase gene of Bacteroides uniformis significantly and selectively increased after fermentation of GPS.

### Example 9 Clinical Trial: Fungal Fiber for Gut Health (FUN4GUT01)

This study compares effects of plant-based fiber vs fungi based fiber on clinical outcomes related to gut function (immunity, emotions, stress) and explores the role of gut microbiome structure and function on individual responses.

The protocol was previously approved by the Independent Ethics Committee for Clinical Pharmacology trials, Foundation for pharmacologic and medicine's studies “Prof. Luis M. Zieher”, Autonomous City of Buenos Aires, Argentina, accredited by the Minister of Health of Buenos Aires City under Res No. 528/2022 GCABA-SSPSGER.

The protocol was registered in Clinicaltrials.gov under ID NCT06404749.
Primary Purpose: Supportive Care
Allocation: Randomized
Interventional Model: Crossover Assignment
Masking: Double (Participant/Investigator)

| Participant Group/Arm | Intervention/Treatment |
|---|---|
| Experimental: FB01 | Dietary Supplement: fungal fiber |
| fungal fiber, 150 mg/day, | · fungal fiber from mycelium |
| oral capsules, 2 weeks | |
| Active | Dietary Supplement: inulin |
| Comparator: inulin | · long chain, degree of 3 |
| inulin, 3 g/day, oral | polymerization DP > 2 |
| capsules, 2 weeks | |

### Primary Outcome:

GastroIntestinal Quality of Life Index (GIQLI), self assessed questionnaire, minimum score 0 (low quality), maximum score 144 (high quality)

### Secondary Outcomes:

Bristol scale and frequency chart. Frequency and consistence of feces, self assessed questionnaire. Range 1 to 7. Optimal value: 4. Values 1-3: different levels of constipation. Values 6-7: different levels of diarrhea
Wisconsin Upper Respiratory Symptom Survey (WURSS41) scale, self assessed questionnaire, range 0 (desired) to 301 (undesired)
Positive and Negative Affect Schedule (PANAS), self assessed questionnaire. Range from 10 (lowest) to 50 (highest) for both positive and negative scale
Work Productivity and Activity Impairment (WPAI), self assessed questionnaire, range 0 (desired) to 100 (undesired)
basal cortisol (fasting)
high sensitivity C Reactive Protein (hs-CRP) (fasting)
fecal calprotectin
fecal and serum (fasting) SCFA

### Other Outcomes:

microbiome metagenomics: gut microbiome taxonomy: abundance, diversity
microbiome metatranscriptomics: expression of gut microbial genes

### Inclusion Criteria:

1. Provision of a signed and dated informed consent form
2. Declared willingness to comply with all study procedures and availability during the study.
3. Female, >37 years, ≤48 years
4. Healthy volunteers who have experienced at least one of the following Gastrointestinal symptoms in the last week: abdominal pain, full stomach, bloating, accidental leakage of intestinal gas, loud belching, bowel movements more frequent than usual, discomfort when eating, with a GIQLI score <121
5. Fiber intake <20 g/day according to R24
6. 18.5≤BMI≤29.9
7. Volunteers permanently living in the Buenos Aires Metropolitan Area for at least 6 months prior to the intervention.
8. Ability to take oral capsules and willingness to comply with intervention regimen of the study.
9. Agreement to comply with lifestyle considerations during the study.

### Exclusion Criteria:

1. History of chronic immune, metabolic or gastrointestinal diseases.
2. History of gastrointestinal surgery.
3. Acute illness within one week prior to screening.
4. Treatment with oral or systemic antibiotics within 3 months prior to screening.
5. Hospitalization within 3 months prior to screening.
6. Consumption of laxatives or antidiarrheals, antispasmodic agents, antacids, anti-inflammatories within three days prior to screening.
7. Current intake of fiber or prebiotics as a supplement, or probiotics as a supplement.
8. Vegan, vegetarian or on a special diet.
9. Pregnancy or breastfeeding.
10. Menopause (absence of menstruation for 12 consecutive months) or postmenopause.
11. Known allergic reactions to components of the study interventions: inulin, beta-glucan, mushrooms, magnesium stearate, microcrystalline cellulose, hydroxypropylmethylcellulose.
12. Treatment with another investigational drug or other intervention in the last 6 months.
13. Receives pharmacological intervention for the treatment of a disease
14. Any disorder that, in the opinion of the investigator, could jeopardize safety of the participant.

### Example 10: Clinical Effects of Oral Intake of 150 Mg GPS Daily for 14 Days, and Differences with Inulin 3 g/d Treatment

After orally consuming GPS as described in EXAMPLE 9, a volunteer aged 40, BMI 21.3, GIQLI score 98, showed an increase of 9 points of the gastrointestinal quality of life measured by the GIQLI index, being the physical dimension the most significant, more precisely the increase in energy and physical resistance. The severity of upper respiratory symptoms common in cold measured by the WURSS44 questionnaire decreased each week, from 21, to 11 to 4 in that order. The decrease was more evident in self-reported tiredness, lack of energy and irritability. The positive affects increased 8 points in the PANAS scale, while the negative affects decreased 9 points. The work productivity impairment was reduced throughout the treatment period from 30 to 0% after treatment, while the impairment to physical activity was reduced from 50% to 0%.

After treatment with GPS, the volunteer showed a Total Serum SCFA increase from 48 uM to 60 μM (FIG. 8) due to an equal increase in propionate and acetate serum levels, as observed in in vitro studies (EXAMPLE 2, FIG. 7). Basal cortisol was reduced from 11.7 to 9.0 μg/dL, while fecal calprotectin and hsCRP remained constant. The Bristol scale did not significantly change, showing predominantly type 1 feces throughout treatment.

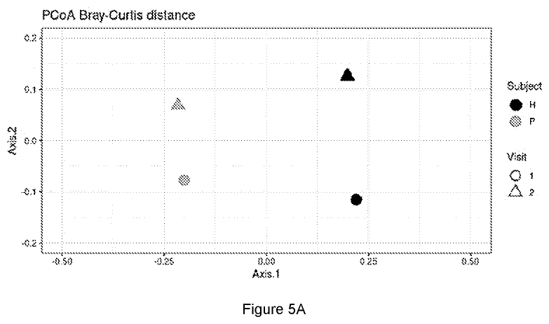

Changes in the fecal microbiome are shown in FIG. 5. Changes in relative frequencies were represented by a decrease in E. coli and Clostridioides difficile (potential pathogens) as observed in in vitro studies, as well as an increase in bacterial diversity (FIG. 6). To note Bifidobacteria frequencies were low in the fecal samples of the volunteer (before and after treatment).

Summarizing, an increase in energy, physical activity, physical resistance, work productivity, and mood balance together with an increase in circulating serum SCFA and slightly decrease of basal cortisol were observed after the oral intake of 150 mg of GPS per day for two weeks. In addition, the treatment caused changes in the fecal microbiome structure, increasing microbial diversity, and reducing relative frequencies of potential pathogens, as seen in in vitro assays.

Responses to inulin (3 g/d) differed considerably from GPS: a volunteer aged 44, BMI 23.5, GIQLI score 114, showed a Total Serum SCFA increase from 49 μM to 57 μM due to an increase in butyrate and secondly propionate, as observed in in vitro studies (EXAMPLE 2, FIG. 7). Basal cortisol, fecal calprotectin and hsCRP increased after treatment from 13.1 to 16.4 μg/dL, 68 to 87 μg/g, and 1.1 to 2.1 ng/L, respectively.

After orally consuming inulin as described in EXAMPLE 9, the gastrointestinal quality of life measured by the GIQLI score increased 11 points, showing the dimension of gastrointestinal symptoms the most significant change, more precisely the decrease in frequency of abdominal pain and bloating. The severity of upper respiratory symptoms, the positive PANAS scale, and work productivity impairment did not show significant changes. The Bristol scale showed a reduction of consistence of feces throughout treatment with inulin, with a clear shift from feces type 3 to type 5 and 6.

Regarding microbiome structure (FIG. 5), relative frequencies of Faecalibacterium prausnitzii were favored, while those of Ruminococcus bicirculans decreased. Fecal samples of this volunteer did not evidence the bifidogenic effect after treatment with inulin although bifidobacteria were detected in the samples. To note, a higher frequency of potential pathogen Klebsiella pneumoniae was observed in fecal samples after treatment with inulin. As observed in FIG. 6, the bacterial diversity decreased after treatment with inulin. Summarizing, inulin treatment caused an improvement in gastrointestinal symptoms and changes in the consistence of feces as expected. However, these effects were not concurrent with a decrease but with an increase in the inflammatory marker hsCRP and an increase in the gut permeability marker calprotectin, while the increase in SCFA levels was lower than that achieved with GPS at a 20× lower dose.

Compared with inulin, GPS showed better performance as prebiotic fiber since it increased microbiome diversity, decreased the frequency of potential pathogenic bacteria, showed higher increase in serum SCFA, inflammatory state measured by hsPCR did not increase and remained at normal levels, gut permeability measured by fecal calprotectin did not increase and remained at normal levels, and immune function, mood and activity and work productivity improved after GPS treatment.

### REFERENCES

Furman D, Campisi J, Verdin E, Carrera-Bastos P, Targ S, Franceschi C, Ferrucci L, Gilroy D W, Fasano A, Miller G W, Miller A H, Mantovani A, Weyand C M, Barzilai N, Goronzy J J, Rando T A, Effros R B, Lucia A, Kleinstreuer N, Slavich G M. 2019. Chronic inflammation in the etiology of disease across the life span. Nat Med. 25:1822-1832. https://doi.org/10.1038/s41591-019-0675-0.
Scheithauer T P M, Rampanelli E, Nieuwdorp M, Vallance B A, Verchere C B, van Raalte D H, Herrema H. 2020. Gut Microbiota as a Trigger for Metabolic Inflammation in Obesity and Type 2 Diabetes. Front Immunol. 11:571731. https://doi.org/10.3389/fimmu.2020.571731.
Bear T L K, Dalziel J E, Coad J, Roy N C, Butts C A, Gopal P K. 2020. The Role of the Gut Microbiota in Dietary Interventions for Depression and Anxiety. Adv Nutr. 11:890-907. https://doi.org/10.1093/advances/nmaa016
Zhang, J., Liu, Y., Tang, Q., Zhou, S., Feng, J., Chen, H. 2019. Polysaccharide of Ganoderma and Its Bioactivities. In: Lin, Z., Yang, B. (eds) Ganoderma and Health. Advances in Experimental Medicine and Biology, vol 1181. Springer, Singapore. https://doi.org/10.1007/978-981-13-9867-4 4
Viceconte F R, Diaz M L, Soresi D S, Lencinas I B, Carrera A, Prat M I, Gurovic M S V. 2021. Ganoderma sessile is a fast polysaccharide producer among Ganoderma species. Mycologia 113:513-524. https://doi.org/10.1080/00275514.2020.1870255.
Thursby E, Juge N. 2017. Introduction to the human gut microbiota. Biochem J. 474:1823-1836. https://doi.org/10.1042/BCJ20160510.
Hrncir T. 2022. Gut Microbiota Dysbiosis: Triggers, Consequences, Diagnostic and Therapeutic Options. 10:578. Microorganisms https://doi.org/10.3390/microorganisms10030578.
Loyd A L, Barnes C W, Held B W, Schink M J, Smith M E, Smith J A, Blanchette R A. 2018. Elucidating “lucidum”: Distinguishing the diverse laccate Ganoderma species of the United States. PLOS One 13: e0199738. https://doi.org/10.1371/journal.pone.0199738.

1. A polysaccharide mixture obtained by fermentation of Ganoderma sessile, wherein said fermentation comprises a step of concentrating a fermentation product by ultrafiltration.

2. The polysaccharide mixture of claim 1, wherein the polysaccharides in the mixture comprise the following monosaccharides, expressed in molar percentage: arabinose 14.3-45.1; xylose 14.9-18.1; mannose 12.6-21.8; galactose 12.6-24.6; glucose 7.0-30.0; fucose 1.2-4.6; rhamnose 0.8-1.6.

3. A composition comprising the polysaccharide mixture of claim 2, further comprising at least one additional excipient selected from a flavoring agent, an antioxidant, a colorant, a mineral, a vitamin, a pH regulator, a protein, a lipid, and a carrier, and any combination thereof, for use in increasing the production of SCFA in the gastrointestinal tract, skin, serum or mucosa of a subject.

4. The composition for use according to claim 3, wherein the composition comprises 150 mg of the polysaccharide mixture of claim 2, 2 mg of sodium stearate, and 148 mg of microcrystalline cellulose.

5. The composition for use according to claim 3 in the treatment or prevention of an illness, disease, infection, or ailment selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

6. The composition for use according to claim 3 in the lost or gain of weight, increase the consume of fiber intake, prevention of aging, achievement of a flat stomach, improvement of the intestinal well-being and quality of life, improvement of mood, decrease in upper respiratory symptoms related to common cold, provision of energetic support to physical, daily and work activity.

7. The composition for use according to claim 5, wherein the polysaccharide mixture is administered at a daily dose of 150-3000 mg.

8. The composition for use according to claim 6, wherein the polysaccharide mixture is administered at a daily dose of 5-100 mg.

9. The composition for use according to claim 8, wherein the polysaccharide mixture is present in the composition in an amount of at least 0.1% w/w.

10. The composition for use according to claim 9, wherein the composition is administered orally.

11. A method for increasing the production of SCFA in the gastrointestinal tract, skin, or mucosa of a subject, comprising the steps of
a) providing a composition comprising a polysaccharide mixture as set forth in claim 2,
b) administering orally an effective amount of the composition of step a) to a subject in need thereof, thereby increasing the production of Short Chain Fatty Acids (SCFA) in the gut, skin or mucosa microbiota of said subject.

12. The method of claim 11, wherein the method is useful for treating or preventing an illness, disease, infection, or ailment selected from gut dysbiosis, gut inflammation, chronic inflammation, coeliac disease, Constipation, Crohn's disease, Diarrhea, Irritable Bowel Syndrome (IBS), Bowel Control Problems (Fecal Incontinence), Gas, bloating, lactose Intolerance, malabsorption, Diverticulosis and Diverticulitis, liver disease, proctitis, ulcerative colitis, hemorrhoids, dyspepsia, short bowel syndrome, anatomic problems of the gastrointestinal tract, skin, or mucosa, colon polyps, cancer, autoimmune diseases, small intestinal bacterial overgrowth, chronic inflammation and related diseases such as cancer, anxiety, depression, type 2 diabetes, asthma, arthritis, dementia, neurodegenerative disorders, high blood pressure, heart disease and stroke, female diseases such as polycystic ovarian syndrome, cancer, pregnancy, menopause, reproductive dysfunction, endometriosis, diseases related to the brain-gut-axis such as autism, depression, anxiety, and schizophrenia.

13. The method of claim 11, wherein the method is useful for losing or gaining weight, increasing the consume of fiber intake, preventing aging, achieving a flat stomach, improving intestinal well-being and quality of life, improving mood, decreasing upper respiratory symptoms related to common cold, providing energetic support to physical, daily and work activity.

14. The method of claim 12, wherein the polysaccharide mixture is administered at a daily dose of 150-3000 mg.

15. The method of claim 13, wherein the polysaccharide mixture is administered at a daily dose of 5-100 mg.

16. A process for the production of the polysaccharide mixture according to claim 2, wherein said process comprises growing Ganoderma sessile in a culture medium, fermenting Ganoderma sessile in a bioreactor at a pH between 5.0-6.0, to obtain a fermentation product, and concentrating a fermentation product by ultrafiltration.

17. The process of claim 16, wherein the culture medium comprises:
i. 20 g/L of a source of carbon selected from glucose, sucrose, maltose, and combination thereof;
ii. a source of nitrogen selected from NH₄Cl at a concentration of 12-18 mg/L, and yeast extract at a concentration of 2 g/L;
iii. an additional component selected from KH₂PO₄ at a concentration of 0.5% w/w, MgSO₄ at a concentration of 0.2% w/w, vitamin B complex and combinations thereof.

18. The process of claim-16 or 17, wherein the carbon source is selected from a mixture of glucose and maltose at a ratio of 1:2 by weight and glucose and sucrose at a ratio of 1:2.
